(19) United States
(12) Patent Application Publication
(10) Pub. No.: US 2026/0076768 A1
(43) Pub. Date: Mar. 19, 2026

(54) OPENING SYSTEM, GRIPPING SYSTEM, AND METHOD

(71) Applicant: Aesculap AG, Tuttlingen (DE)

(72) Inventors: Arne Jansen-Troy, Stockach (DE); André-Manuel Eberhardt-Fonseca, Stuttgart (DE); Allan Maas, Konstanz (DE); Tobias Koller, Tuttlingen (DE)

(21) Appl. No.: 19/331,105

(22) Filed: Sep. 17, 2025

(30) Foreign Application Priority Data
Sep. 19, 2024 (DE) 10 2024 126 886.7

(51) Int. Cl. A61B 90/57; B25J 17/02

(52) U.S. Cl. CPC A61B 90/57; B25J 17/0283

(57) ABSTRACT

An opening system opens and/or unlocks a medical, in particular surgical, instrument with ring handles. The opening system includes a base and two axles arranged on the base. The axles are movable relative to each other by an actuator. The axles are arranged on flat base elements that are at different distances from the base. Ring handles of a surgical instrument can be threaded onto the axles. Two swivel clamping devices have clamping elements for clamping the ring handles threaded onto the axles to the base elements.

### CROSS-REFERENCE TO RELATED APPLICATION

This application claims priority under 35 U.S. C. § 119 to German Application No. 10 2024 126 886.7, filed on Sep. 19, 2024, the content of which is incorporated by reference herein in its entirety.

### FIELD

The present disclosure relates to an opening system and a gripping system with such an opening system. It also relates to a method for operating the gripping system.

### BACKGROUND

Carrying sterile goods containers and lifting or loading and unloading them into sterile goods container transport trolleys and shelves at very high or low positions, as well as removing the sieve baskets from the containers, continues to be tiring work that is harmful to the health (especially the back) of employees in a medical device reprocessing unit (AEMP) over the long term. Opening or unlocking medical instruments by hand is also a strenuous and tiring task. Several technical solutions have therefore already been proposed for the automated transport, loading, unloading, and handling of sterile goods containers and sieve baskets.

Research approaches to instrument separation based on object recognition of medical instruments are severely limited in practice. The availability of CAD data for all instruments to be sorted, or the assumption that instruments will arrive at an AEMP clean and in a closed state, is unrealistic for instrument separation on the unclean side. A gripping system for sorting medical instruments from a sieve basket must be able to grab instruments stacked on top of each other separately from one another. In addition, medical instruments should be able to be successfully placed in the desired location in order to avoid unnecessary process steps or obstructing the next process step.

EP 3 923 850 A1 discloses an unlocking device for unlocking locking devices comprising cooperating locking elements of at least two medical ring instruments, wherein the unlocking device comprises a first receiving body with at least two ring receptacles for picking up first rings of the at least two ring instruments, and wherein the unlocking device comprises an unlocking body with at least two unlocking elements interacting with second rings of the at least two ring instruments, wherein the unlocking body and the receiving body are arranged so as to be movable relative to each other in an actuating direction. The medical instruments must be opened for insertion into the unlocking device in such a way that they can be picked up by the unlocking device, as it cannot be adjusted to the opening angle of the medical instruments.

### SUMMARY

The present disclosure is therefore based on the task of providing an opening system for automatically opening and/or unlocking a medical instrument. The present disclosure is further based on the task of providing a gripping system that performs the automated handling of medical instruments. The present disclosure is further based on the task of providing a corresponding operating method.

With regard to the opening system, the above-mentioned task is solved by an opening system that has a base and two axles arranged on the base and movable relative to each other by means of an actuator, which are each arranged on flat base elements that are at different distances from the base. The opening system further comprises two swivel clamping devices, each of which comprises a clamping element for clamping a ring handle (i.e. a ring grip or loop) threaded onto an axle to the base element.

The present disclosure is based on the idea that medical instruments with ring handles, which are usually opened and closed using the grips, must have a specific opening angle for further processing, in particular for cleaning and sterilization. In addition, these medical instruments usually have a locking mechanism that must be released in order to set the medical instrument to a specific opening angle.

It has now been recognized that these tasks can be performed with the aid of an automated opening system, which independently performs both the opening and unlocking of the medical instrument without manual intervention. Such an opening system can be integrated into a gripping system, enabling automated processing of medical instruments from delivery of the sterile goods containers to dispensing of the medical instruments on the clean side by the cleaning and disinfection devices (RDGs).

The medical instrument in question is specifically a surgical instrument, but may also be an orthopedic or neurological instrument.

Advantageously, the respective swivel clamping device is designed as a swivel clamping cylinder. The swivel clamping cylinder can enable swiveling and movement of the clamping element toward the base and in the opposite direction according to the pneumatic or hydraulic principle. Alternatively, an electric drive can also be provided.

The respective swivel clamping device advantageously comprises the base element, which can be moved by means of a drive to enable the ring instrument to be opened in a respective downward or upward direction.

The swivel clamping cylinders can be force-controlled, but it is preferable to provide an additional force sensor for control purposes. The control can be carried out via the pressure in the system or by a force torque sensor, but preferably via a strain gauge integrated into the respective base surface. The control and regulation unit receives information on the direction of movement of the swivel clamping device or the swivel clamping cylinder from the measurements of the proposed force sensor and/or from the optical system.

The difference in level between the two base surfaces, i.e., also the difference in their distance from the base, is preferably at least 5 mm, particularly preferably 10 mm to 15 mm.

In a preferred embodiment, a rail is arranged on the base, on which a carriage can be moved, on which one of the axles and a swivel clamping device are arranged. The distance between the two axles can be varied by means of the carriage, so that the medical instrument can be opened and closed when the ring handles are engaged with one axle and clamped in place.

The carriage is preferably movable along the rail by means of a spindle rotatable by the actuator.

The actuator of the opening system preferably comprises an electric drive and a gearbox.

With regard to the gripping system, the above-mentioned task is solved by the present disclosure by means of a gripping system that includes a gripper for lifting and placing the medical instrument, an object recognition device and an opening system as described above, as well as a control and regulation unit. The object recognition device is designed to recognize a medical instrument with ring handles, wherein the gripping system is designed to insert the medical instrument with ring handles into the opening system.

The object recognition device advantageously comprises a classifier, which is designed in particular as a neural network, for classifying the medical instrument into the classes “has ring handles” and “does not have ring handles”. Only if the medical instrument has ring handles should it be inserted into the opening system.

The classifier is preferably designed to recognize a locking mechanism of the medical instrument, wherein the control and regulation unit controls the gripper on the basis of the recognized locking mechanism in such a way that it is inserted into the opening system in an unlocked orientation.

The gripping system advantageously comprises a unit having six (6) degrees of freedom (“6-DOF unit”) on which the opening system, in particular the base of the opening system, is mounted.

The gripping system advantageously comprises a 6-DOF articulated arm robot on which the gripper is mounted. The 6-DOF unit and the 6-DOF articulated arm enable very flexible positioning and alignment of the gripper relative to the opening system.

With regard to the method, the above-mentioned task is solved according to the present disclosure by a method that includes the steps of gripping a medical instrument and placing the instrument on a placement surface and/or positioning the instrument in front of a reference surface, optically detecting the contours of the medical instrument and recognizing whether the medical instrument has ring handles, and if this is the case, placing the medical instrument in the opening system, and opening and/or unlocking the medical instrument through the opening system, as well as repeated gripping the medical instrument and placing the medical instrument at a predetermined location.

The step of gripping a medical instrument and placing the medical instrument on a placement surface and/or positioning the medical instrument in front of a reference surface comprises the step that the medical instrument must be used to correctly recognize the outlines/geometry without placement in front of a reference surface (e.g., solid green surface) while still being positioned by the robot arm or gripper is/remains.

The medical instrument can then either be placed and picked up again by another robot arm or gripper (or the same one), possibly with the gripping position corrected to the calculated gripping coordinate, or it can be transferred directly to another robot arm without placement, which then grips/picks up the medical instrument at the gripping coordinate.

Advantageously, the contours of the medical instrument are recognized using an edge recognition algorithm. The Canny edge recognition algorithm is particularly preferred for this purpose.

In a preferred embodiment, features on the medical instrument are recognized with the aid of the object recognition device and an output is generated based thereon. These features include data matrix codes, barcodes, labeling, etc. This allows the item number and lot number to be recognized even on the dirty side, and a comparison with the packing list can be used to report whether all medical instruments are present. The message is preferably reflected in the product management system, i.e., via an interface to the user, or it is automatically added. This is currently only possible when packing.

Gripping takes place along a predetermined grid. For this purpose, it is advantageous to specify points on a surface in the x and y directions and then in the z direction once a surface has been completely traversed. At the end, check that the sieve has been emptied. This means that the first level (i.e., the top level) is traversed first, followed by a second, lower level, so that when moving in the x and y directions, the gripper does not displace any medical instruments and thereby wedge them together and/or damage the medical instruments.

The advantages of the present disclosure lie in particular in the fact that the opening system enables automated opening and unlocking of medical instruments with ring handles, thereby reducing the workload of medical personnel.

The gripping system closes a gap in existing automation, enabling fully automated handling of instruments from the unclean to the clean side. The gripping system can be used on the unclean side of an AEMP (medical device reprocessing unit) and as a subsystem of a robotic instrument separation system to automate all processes within the sterile goods cycle, from the delivery of contaminated medical instruments in a sterile goods container to their cleaning and disinfection.

The method described allows medical instruments classified as “no ring handle” and “ring handle” to be picked up from a placement surface in a targeted manner so that they can then be placed according to their class and further manipulated. Medical instruments with a ring handle can be opened automatically at the ideal opening angle in accordance with the specifications for the respective medical instrument. These specifications are stored digitally and are then passed on to the axle control system when the instrument geometry is recognized. This allows the medical instruments to be prepared for optimal cleaning, especially automated cleaning.

### BRIEF DESCRIPTION OF THE DRAWINGS

An exemplary embodiment of the present disclosure is explained in more detail with reference to the accompanying drawings, of which:

FIG. 1 shows a gripping system in a preferred embodiment;

FIG. 2 shows an opening system in a preferred embodiment;

FIG. 3 shows a placement surface with a pair of scissors;

FIG. 4 shows the pair of scissors with calculated geometric dimensions, and

FIGS. 5A-5D show sections of a flowchart of a method in a preferred embodiment.

Identical parts are marked with the same reference symbols in all figures.

### DETAILED DESCRIPTION

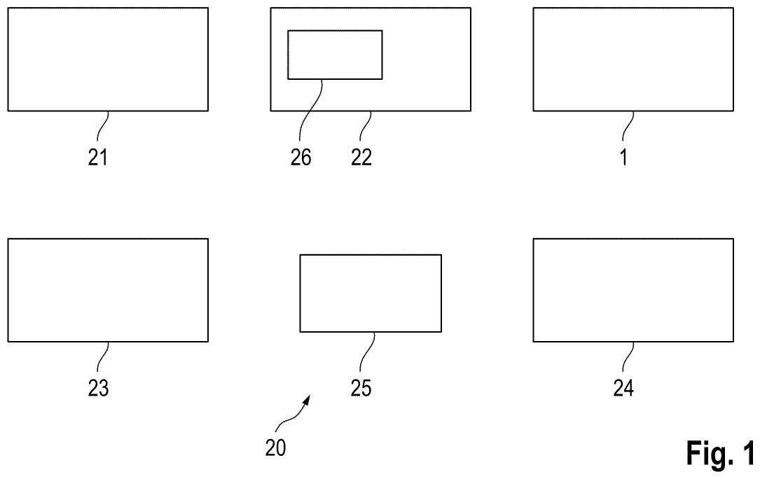

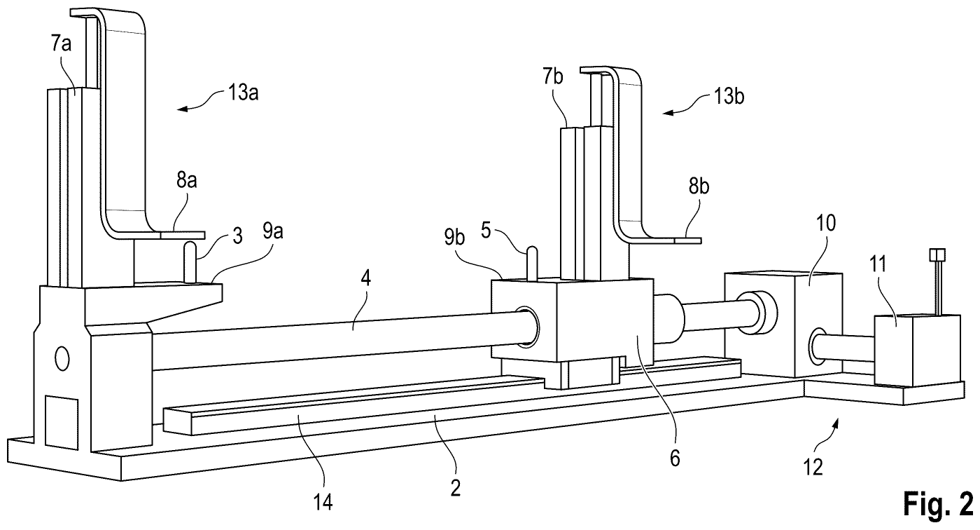

A gripping system 20 schematically illustrated in FIG. 1 comprises an electromagnetic gripper 21 for lifting and placing a medical, in particular surgical, instrument 38. The gripping system 20 further comprises an object recognition device 22 and an opening system 1, which is described below in connection with FIG. 2. The gripping system 20 further comprises a 6-DOF unit on which the opening system 1 is mounted, and a 6-DOF articulated arm robot 24 on which the gripper 21 is mounted.

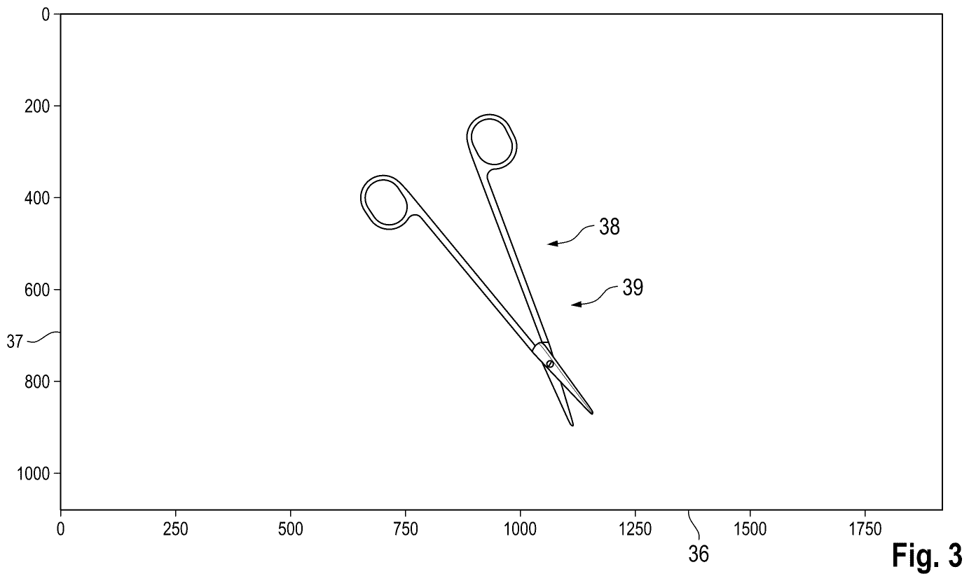

The object recognition device 22 is designed to recognize a medical instrument 38 (see FIG. 3) that is equipped with ring handles and, for this purpose, has a classifier 26, which is designed as a neural network that has been trained by supervised learning using images of medical instruments with and without ring handles. The gripping system 20 is designed to place the medical instrument 38 with ring handles into the opening system 1. The gripping system 20 has a control and regulation unit 25, which can be designed as a central control and regulation unit or can comprise several control and regulation units assigned to the individual system components.

FIG. 2 shows an opening system 1 for ring instruments in a preferred embodiment. The opening system has a base 2. The base 2 can be mounted on a 6 DOF unit (6 degrees of freedom unit) that is preferably spatially and automatically movable (not shown). Alternatively, the opening system 1 can be stationary, in which case the opening system 1 is preferably located within the cell and in the working area of the electromagnetic gripper 21.

The opening system 1 comprises two automated axles 3, 5 that can be moved relative to each other, which are cylindrical in design and located on the base 2. The two axles 3, 5 can be set to the distance between the two ring handles or the two legs of a ring instrument 38 to be manipulated (see FIG. 3) in its current state (if applicable, the closed and thus detent state or the fully open state or positions between these end positions) (the calculation of the distance between the two ring handles is explained below 2), which can be moved or positioned by the electromagnetic gripper 21 described above.

One of the two axles 3, 5 can be designed as non-movable relative to the base 2 and thus maintain a relatively fixed position. Since medical instruments such as artery clamps in particular have a detent mechanism, the opening system 1 is designed to enable the detent ring handles to be opened.

For this purpose, the axle 5, which can be positioned by means of an actuator 12, which in this case is designed as a spindle drive 4, can be moved linearly on a carriage 6 on a rail 14 fixed to the base 2. In the present case, an electric drive 11 is provided, which drives a spindle 4 via a gear 10 using an encoder. The encoder can be used to set the desired position of the two axles 3, 5 relative to each other via the electric drive 11 by rotating the spindle 4.

The opening system 1 comprises two swivel clamping devices 13a, 13b, which are designed here as swivel clamping cylinders 7a, 7b and are closed as soon as the medical instrument is threaded onto the two axles 3, 5 with the two ring handles. The two ring handles on the axles 3, 5 are pressed onto the base surfaces of the axle receptacles 9a, 9b via two clamping elements 8a, 8b mounted on the swivel clamping cylinders 7a, 7b. A height offset between the base surfaces 9a and 9b, together with the clamping of both ring handle rings at different levels of the base surfaces 9a and 9b, ensures that the detent is released, for example in the case of artery clamps.

The two grip rings are clamped in two parallel planes between a base surface 9a or 9b and a clamping element 8a or 8b. Since the two clamping planes are arranged parallel to each other but at different heights above the base, the two branches/legs of the gripping elements are elastically deformed against each other, so that a lock (or detent) provided between these branches is disengaged. Once the medical instruments 38 are clamped and the detent mechanism is open, the two axles 3, 5 can be moved apart relative to each other to set the optimum opening angle (e.g., 90°). This allows for optimal cleaning afterwards. It is proposed that, in addition to recognition or navigation for threading the ring instruments, the optimum opening angle for each ring instrument or item be stored and set via a programmable logic controller (not shown).

The clamping elements 8a, 8b are advantageously swiveled via the swivel clamping cylinders 7a, 7b so that the rings of the medical instruments 38 can be pushed onto the axles 3, 5. The respective swivel clamping cylinder is then swiveled back (position as shown in FIG. 1) and subsequently clamps the ring of the medical instrument 38 opposite the respective base surface 9a or 9b. The swivel clamping cylinders 7a, 7b are usually force-controlled, but it is recommended to use an additional force sensor for control purposes. This can be done via the pressure in the system or via a force torque sensor, but ideally via a strain gauge integrated into the respective base surface 9a, 9b. It is recommended that the difference in height between the two base surfaces 9a and 9b be at least 5 mm, but preferably 10 mm to 15 mm.

To calculate the distance between the two ring handles after positioning on the placement surface and subsequently manipulating the instrument alignment, a dark background is ideal for object recognition and, in particular, for edge recognition using the state-of-the-art Canny algorithm. A bright background, combined with a lack of contrast on highly reflective instruments, can cause the camera to overexpose some shots.

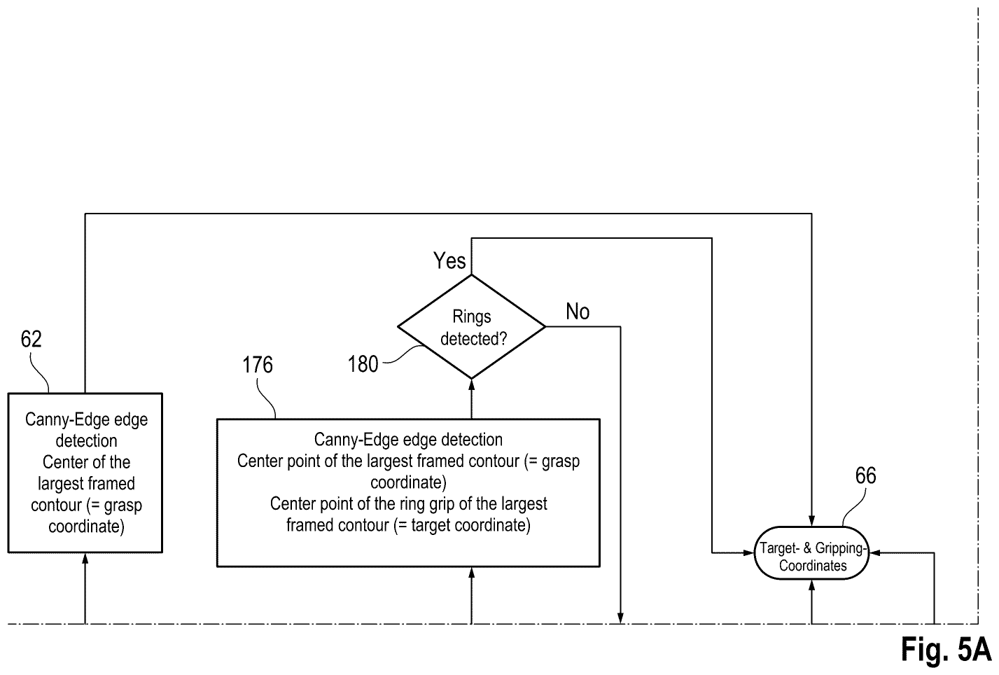

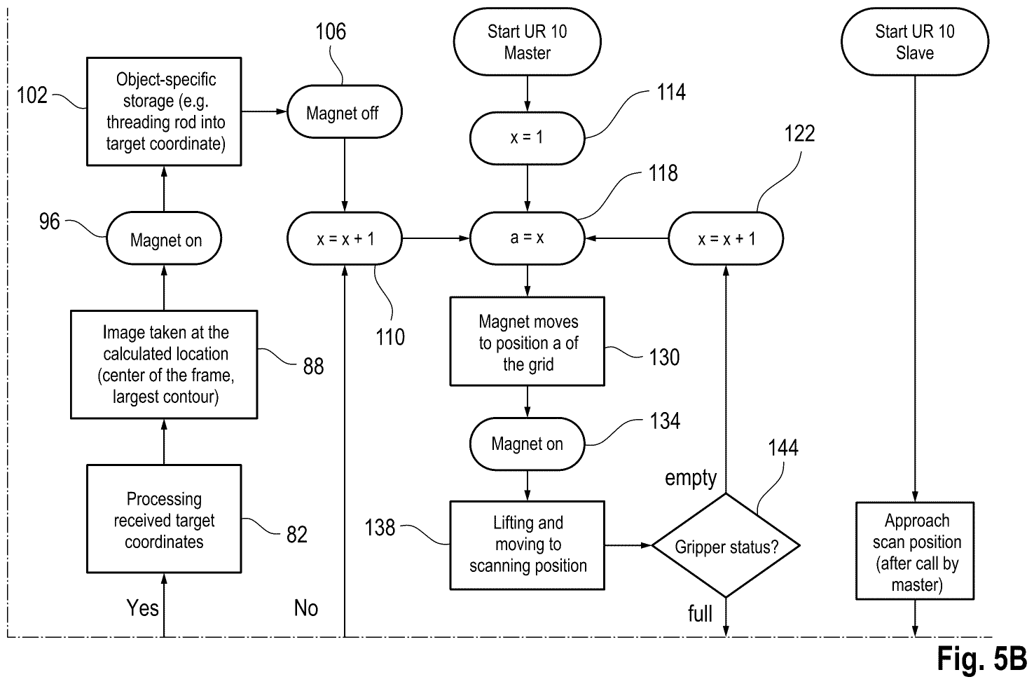

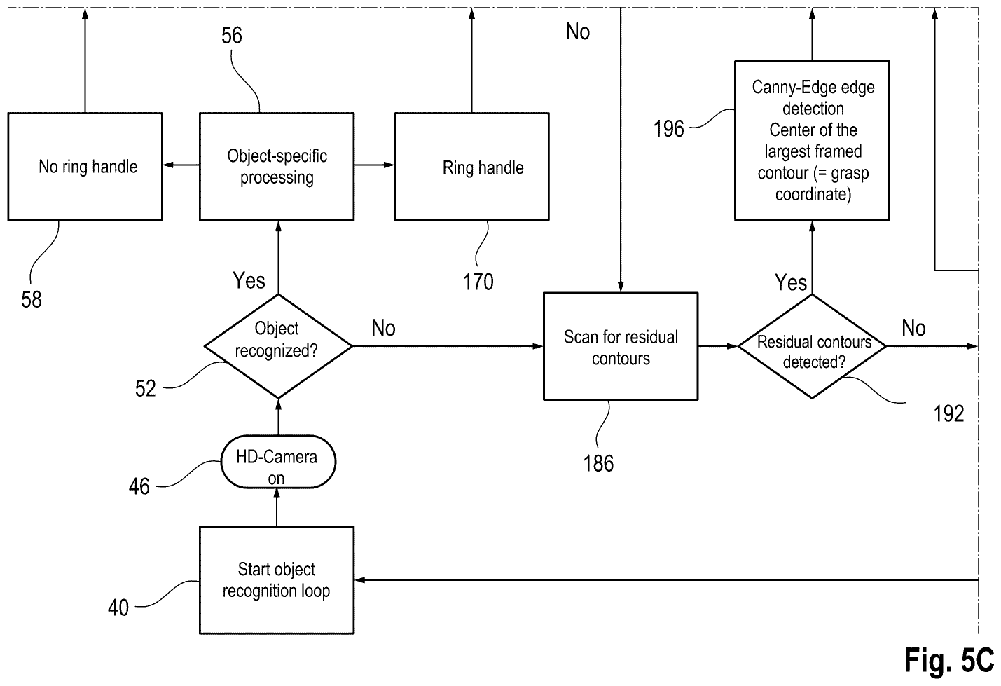

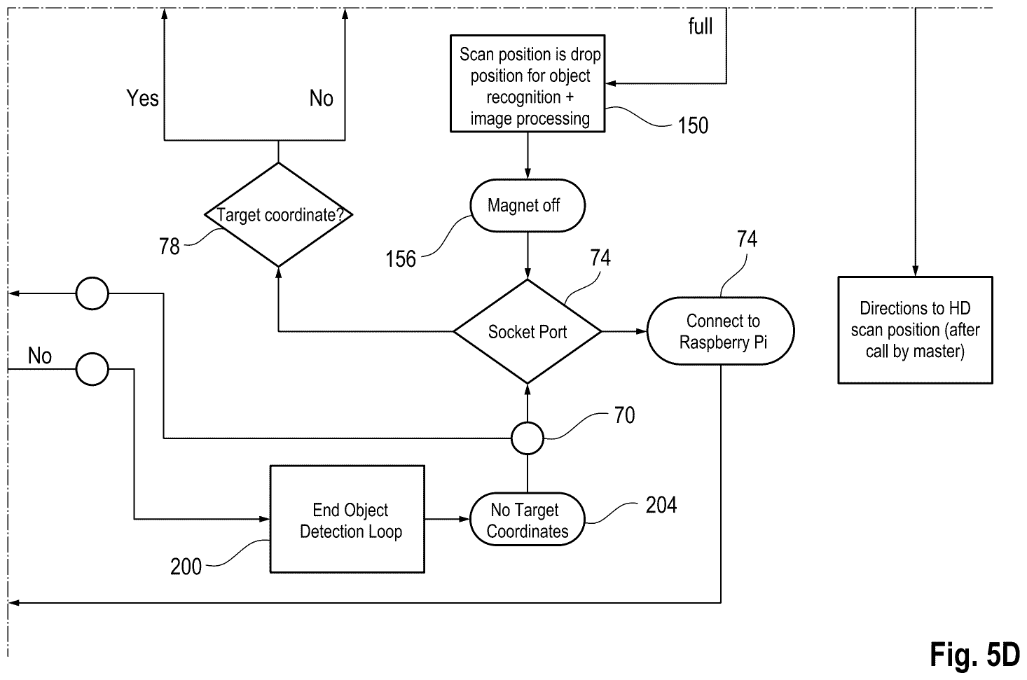

FIGS. 5A-5D schematically show sections of a flow chart of a method for operating the gripping system 20 in a preferred embodiment. In principle, it is necessary to grip various medical instruments, which are divided into the instrument classes “ring handle” and “no ring handle” as a prerequisite for the underlying method. Medical instruments 38 generally have a ring handle (e.g., pair of scissors) or a mechanism not connected to ring handles (e.g., tweezers) that allows them to be opened and closed. The kinematics for opening the medical instruments 38 is crucial for successful automated separation and cleaning of medical instruments 38.

A medical instrument 38 is placed on a placement surface by means of a rigid grid (depending on the size of the sieve basket) using an electromagnetic gripper 21 after being “gripped from the box”. This step, up to the point where the part is placed on the placement surface, is performed without any specific calculation of the gripping coordinates. However, in order to place a medical instrument 38 in a defined position with a specific orientation, it is necessary to calculate the gripping coordinates and to know the rings of a medical instrument 38 of the “ring handle” class in order to open it using the mechanism described above.

The coordinates can also be detected without placing the medical instrument 38 by positioning the medical instrument 38 in front of a reference surface while still gripping it.

The method begins with object recognition in step 40, where the object recognition loop is started. In the first terminal, 46, the placement surface is scanned by recording of the placement surface from a bird's-eye perspective with a webcam. The image plane is parallel to the object plane or placement surface. The classifier 26, a neural network, assigns the placed medical instrument 38 to one of the two classes “ring instrument” and “non-ring instrument”. The neural network used for this purpose is specially trained for the two classes and is then able to assign medical instruments with and without ring handles to the two previously defined classes.

A decision 52 determines whether the medical instrument 38 has been recognized. If this is the case, the method continues in step 56, in which object-specific further processing of the recognized medical instrument 38 is initiated.

Medical instruments 38 without ring handles do not usually need to be opened at a special opening angle for cleaning. Placing them on a cleaning basket or other conveyor mechanism is sufficient for further transport or the actual cleaning of the medical instruments 38. Since the method for repeated picking up of medical instruments 38 without a ring handle is correspondingly simpler, this branch of the method is described first, beginning in step 58.

Based on the calculation of the gripping coordinates of medical instruments 38 without a ring handle, the proposal for calculating the gripping coordinates of medical instruments 38 with a ring handle is then described, along with how these are specifically aligned so that they can be opened using the mechanism described above.

In step 62, the captured image is processed from a bird's eye view using a Canny edge recognition algorithm. The result is a one-pixel-thick edge representation of the contours of the medical instrument 38. Edge pixels of the medical instrument 38 are preferably displayed in black, all other pixels in white. A rectangle is drawn around the largest continuous contour (the outer contour of the placed medical instrument 38). In the next sub-step of step 62, the center point of the rectangle is calculated in pixel coordinates. In order to be able to pick up the medical instrument 38 again with the gripper 21 in a later step, the first available edge pixel is searched for in both the horizontal and vertical directions. Once this has been found, it is defined as the new gripping coordinate for repeated picking up of the placed medical instrument 38 in pixel coordinates.

The gripper 21 is advantageously mounted on the 6-DOF articulated arm robot 24, so the calculated pixel coordinates must first be transformed into robot coordinates (tool center point (TCP) of the robot). The transformation is performed using a previously stored corner point of the image section of the placement surface (for example, the upper left corner) in robot coordinates and the vector addition from the corner to the new gripping coordinate. The vectors are known in a metric unit (millimeters) by converting the distance into pixels using a previously calculated constant conversion factor.

The target and gripping coordinates are known in terminal 66, so the method continues to node 70. At the start of the method, in step 74, a data connection to a processing unit is set up, which is started in a terminal 74. The method then begins the loop described above for object recognition in step 40.

Starting from step 74, a decision 78 is made to check whether target coordinates are available. If this is the case, the method continues in step 82, in which the target coordinates are processed. Here, the respective calculated pixel coordinates are first transformed into robot coordinates (tool center point (TCP) of the robot).

The gripper 21 picks up the medical instrument 38 in a single step 88 at the calculated coordinates (center point of the outer contour) and places the medical instrument 38 in a class-specific position. In terminal 96, the magnet of gripper 38 is switched on. In step 102, object-specific placement takes place, for example, threading a rod at the target coordinate. In terminal 106, the magnet of gripper 38 is switched off. The variables x and a are the transfer values of the coordinates, which are referenced to the TCP (Tool Center Point) plane.

If there are no target coordinates in decision 78, the method continues in terminals 110, 118 to step 130, in which the magnet moves to position a of the grid. In terminal 134, the magnet is switched on and, in a step 138, a medical instrument 38 is lifted and moved to a scanning position. In decision 144, the gripper status is checked. If gripper 21 is empty (i.e., moving to the scan position with a lifted instrument was unsuccessful), the method continues in terminal 122. Otherwise, the method branches to step 150. The scan position corresponds to the drop position for object recognition and image processing. In a terminal 156, the magnet is switched off for this purpose.

If, during the first rigid “gripping into the box,” several medical instruments 38 are gripped up by the electromagnetic gripper 21 described above and placed on the placement surface, a new scan of the placement surface is performed without another “gripping into the box” after the previously described picking up of the recognized instrument 38. The algorithm described above is repeated until no medical instruments 38 are detected on the placement surface. The Canny edge algorithm is also used to recognize whether a medical instrument 38 is still lying on the placement surface. If no edges are recognized, the placement surface is recognized as empty.

If the neural network recognizes a medical instrument 38 of the “ring handle” class (step 56), an extended calculation is performed, which begins in step 170. This is described in the following section. Similar to the calculation for the “no ring handle” class, for instruments in the ring handle class, edge recognition is first performed in step 176 using the Canny edge algorithm and the largest contour is calculated.

FIG. 2 shows an unprocessed representation of the optically detected placement surface on the x axis 36 and the y axis 37, with pixel coordinates plotted on each axis. A medical instrument 38 is lying on the placement surface, which in this case is a pair of scissors 39 and therefore a ring gripping instrument.

Unlike medical instruments 38 without ring handles, however, medical instruments 38 with ring handles are to be opened by means of the opening system 1 described in connection with FIG. 2. In addition to the gripping coordinates, the following information is generally required for repeated picking up:
center points of both circles (ring handles) in robot coordinates
distance between the two center points to move the two axles of the mechanism described above to the desired distance
overall orientation of the instrument in the plane (joint of the medical instrument 38 above/below the ring handles) in order to be able to open a detent, e.g., on artery clamps (with the aid of the swivel clamping cylinders 7a, 7b)
new target position of the ring handles for a defined opening angle at the specific position for medical instruments 38 in the “ring handle” class.

The two ring handles are detected using an open source tool, such as the tool available under the registered trademark OPENCV with the HoughCircles() function provided. This is freely accessible code for recognizing circles in images. A parameter set can be used to specify a range of radii to be recognized. Since ring handles on medical instruments 38 usually have a similar radius, the necessary range of possible radii is adjusted iteratively using the parameter set. The HoughCircles() command requires a grayscale image. Since the image has already been edge-segmented using the Canny edge recognition algorithm, this requirement is fulfilled.

Ring handles are generally not round, but elliptical in shape. However, calculating and recognizing ellipses is much more complex than calculating and recognizing circles. Therefore, the partial circles of the upper and lower halves of both ellipses are recognized as radii and the center point of the ellipses is approximated accordingly. This simplification can be made because the respective mechanical axle does not have to be positioned exactly at the center point of the ellipses; instead, positioning within the ellipse is sufficient to open the medical instruments 38.

Now, the distance between the two center points of both elliptical ring handles in the image plane is known. Based on knowledge of these two points, the orientation of the medical instrument 38 on the placement surface and the new gripping coordinates for repeated picking up with the electromagnetic gripper 21 are calculated. To do this, first draw a straight line connecting the two center points. The angle of these connecting lines relative to the horizontal plane provides initial information about the orientation of the medical instrument 38. In the next step, search for the first available edge pixel (black pixel) at right angles to the line connecting the two points, halfway between the line and the upper and lower edges. With a ring handle instrument 38 opened minimally, the first pixel found is located near the joint. This calculated point is well suited for repeated picking up of the medical instrument 38 in a stable manner using an electromagnetic gripper 21.

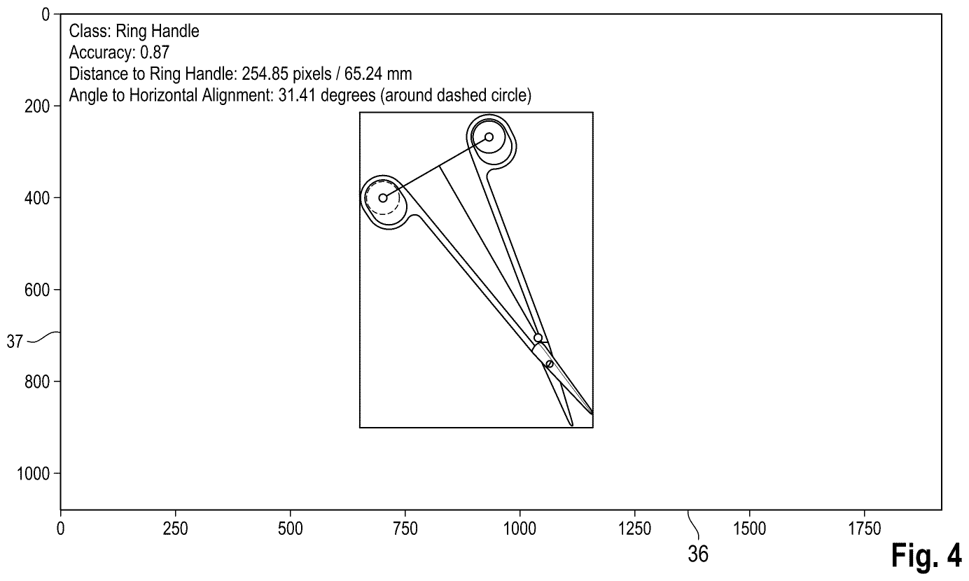

The calculation is shown graphically in FIG. 4, in which the recognized corners and the surrounding contour are also easily recognized.

A decision 180 checks whether the rings were recognized by the procedure described above. If this is the case, the two points within the two elliptical ring handles, the orientation angle of the medical instrument 38 on the surface, and a new gripping coordinate in pixel coordinates are now known, and the method continues in terminal 66. The information as to whether the new gripping coordinate lies above or below the straight line also indicates where the tip of the medical instrument 38 is located. All information is therefore available to pick up the instrument with the electromagnetic gripper 21 and move axles 3 and 5 to the calculated distance (length of the connecting lines) for opening via the ring handles and position them within the ellipses. The conversion of pixel coordinates into robot coordinates is performed in the same way as in the “no ring handle” class.

If no rings were recognized in decision 180, the method branches to step 186, in which a scan for residual contours is performed. If no residual contours are recognized, a new sieve basket or instruments are supplied and the algorithm starts again. A decision 192 checks whether residual contours have been recognized. If this is the case, the method branches to step 196, in which edge recognition is performed using the Canny edge algorithm and the center point of the largest framed contour is determined, which represents the target coordinates for gripper 21. The method then continues with terminal 66. If no residual contours were recognized in decision 192, the object recognition loop is terminated in step 200, and no target coordinates are available in terminal 204. The master-slave architecture shown in FIG. 5 represents the communication between the two robots and the sensor system.

### LIST OF REFERENCE SIGNS

1 Opening system
2 Base
3 Axle
4 Spindle
5 Movable axle
6 Carriage
7a, 7b Swivel clamping cylinder
8a, 8b Clamping element
9a, 9b Base element of the axle receptacle
10 Gearbox
11 Electric drive
12 Actuator
13a, 13b Swivel clamping device
14 Rail
20 Gripping system
21 Gripper
22 Object recognition device
23 6-DOF unit
24 6-DOF articulated arm robot
25 Control and regulation unit
26 Classifier
36 x axis
37 y axis
38 Medical instrument
39 Pair of scissors
40 Step
46 Terminal
52 Decision
56 Step
58 Step
62 Step
66 Terminal
70 Junction
74 Step
78 Decision
82 Step
88 Step
96 Terminal
102 Step
106 Terminal
110 Terminal
114 Terminal
118 Terminal
122 Terminal
130 Step
134 Terminal
138 Step
144 Decision
150 Step
156 Terminal
170 Step
176 Step
180 Decision
186 Step
192 Decision
196 Step
200 Step
204 Terminal

1. An opening system for opening and/or unlocking a medical instrument with ring handles, the opening system comprising:
a base;
an actuator;
a first axle and a second axle arranged on the base and movable relative to each other via the actuator, the first axle arranged on a first flat base element and the second axle arranged on a second flat base element, the first flat base element and the second flat base element being spaced from the base at different distances;
a first swivel clamping device comprising a first clamping element and a second swivel clamping device comprising a second clamping element,
the first clamping element configured for clamping a first ring handle threaded onto the first axle to the first flat base element, and
the second clamping element configured for clamping a second ring handle threaded onto the second axle to the second flat base element.

2. The opening system according to claim 1, wherein the first swivel clamping device and the second swivel clamping device are each designed as a swivel clamping cylinder.

3. The opening system according to claim 1, wherein the first flat base element and the second flat base element are movable by a drive.

4. The opening system according to claim 1, further comprising a rail arranged on the base and a carriage that is movable on the rail, wherein the second axle and the second swivel clamping device are arranged on the carriage.

5. The opening system according to claim 4, wherein the carriage is movable along the rail by a spindle that is rotatable by the actuator.

6. The opening system according to claim 1, wherein the actuator comprises an electric drive and a gear.

7. A gripping system for picking up and handling medical instruments, the gripping system comprising:
the opening system according to claim 1;
a gripper for lifting and placing the medical instrument;
an object recognition device; and
a control and regulation unit,
wherein the object recognition device is configured to recognize a medical instrument with ring handles, and
wherein the gripping system is configured to insert the medical instrument with ring handles into the opening system.

8. The gripping system according to claim 7, wherein the object recognition device comprises a classifier configured to classify the medical instrument into a first class corresponding to an instrument that has ring handles and a second class corresponding to an instrument that does not have ring handles.

9. The gripping system according to claim 8, wherein the classifier comprises a neural network.

10. The gripping system according to claim 8, wherein the classifier is configured to recognize a locking mechanism of the medical instrument, and when the locking mechanism is recognized, the control and regulation unit controls the gripper in such a way that the medical instrument is inserted into the opening system in an unlocked orientation.

11. The gripping system according to claim 7, further comprising a unit having six degrees of freedom on which the base of the opening system is mounted.

12. The gripping system according to claim 7, further comprising an articulated arm robot having six degrees of freedom on which the gripper is mounted.

13. A method for operating the gripping system according to claim 7, the method comprising the steps of:
gripping a medical instrument and placing the medical instrument on a placement surface and/or positioning the medical instrument in front of a reference surface;
optically detecting contours of the medical instrument and recognizing whether the medical instrument has ring handles;
positioning the medical instrument in the opening system and opening and/or unlocking the medical instrument through the opening system when the gripping system recognizes that the medical instrument has ring handles; and
gripping the medical instrument and placing the medical instrument in a predetermined location.

14. The method according to claim 13, wherein the step of gripping the medical instrument and placing the medical instrument in the predetermined location is repeated.

15. The method according to claim 13, wherein contours of the medical instrument are recognized using an edge recognition algorithm.

16. The method according to claim 13, wherein the object recognition device recognizes features on the medical instrument and generates an output based on the features recognized on the medical instrument.

17. The method according to claim 13, wherein the step of gripping the medical instrument takes place along a predetermined grid.
